(19) United States
(12) Patent Application Publication
(10) Pub. No.: US 2010/0069922 A1
(43) Pub. Date: Mar. 18, 2010

(54) Suture Cutter with Replaceable Blade

(76) Inventor: David L. Kaufman, Gainesville, FL (US)

Correspondence Address: PHILIP H. BURRUS, IV, 460 Grant Street, Atlanta, GA, 30312, US

(21) Appl. No.: 12/233,357

(22) Filed: Sep. 18, 2008

(51) Int. Cl. A61B 17/10

(52) U.S. Cl. 606/138

(57) ABSTRACT

An apparatus (100) for cutting sutures (601) is provided. The apparatus (100) includes a handle (101) and an extension member (105) extending therefrom. The extension member (105) includes a first extension rail (103) coupled fixedly with the handle (101) and a second extension rail (104) that is configured to slide along the first extension rail (103) when cutting a suture (601). A detachable blade (108) is coupled to the second extension rail (104) and can be replaced when a compliance member (114) is moved such that the second extension rail (104) is able to pivot away from the first extension rail (103). A user actuates a push button (110) that causes the second extension rail (104) to move such that the detachable blade (108) passes into a blade receiving aperture (207), thereby severing a suture (601) engaged in the suture hook (106).

### BACKGROUND

1. Technical Field

This invention relates generally to surgical cutting tools, and more particularly to a surgical cutting tool suitable for cutting, for instance, sutures that includes the ability to replace the blade.

2. Background Art

Sutures are a necessary part of almost all surgical procedures. When a surgeon cuts skin or tissue to perform a surgical operation, sutures, or “stitches”, are typically used to close the incision. Sutures are formed when the surgeon threads a strong, hypoallergenic thread through the patient's tissue so that the skin, internal organs, and tissues are held together. Sutures hold the tissue in a closed position thereby permitting the body to repair the incision. When the surgeon is suturing the patient, excess thread must be neatly cut about the suture's knot. For some suture material, once the tissue has substantially healed, the doctor must then cut each suture and pull the suture out so that the healing process can be completed. Sutures are made using both absorbable material, which breaks down naturally over time, and non-absorbable material, which offers increased tensile strength and durability but must be cut during removal.

Any time a non-absorbable material is used for a suture, the suture must be cut and physically removed once the afflicted area has adequately healed. Regardless of the type of material used, excess material must be cut from the knot when the sutures are installed. Sutures are generally cut with standard surgical scissors. For specialized procedures, such as arthroscopic surgery, some specialized types of suture cutters have also been developed. Examples of such specialized suture cutters include those disclosed in US Published Pat. Application No. 2006/0212045 to Schilling et al. and US Published Pat. Application No. 2007/0106310 to Goldin et al.

Both surgical scissors and these specialized suture cutters operate by using a dual-edge, scissor-type engagement where two sharpened edges pass across each other to cut a suture or other material. One problem with these types of cutting devices is—as anyone who has used household scissors will relate—once either of the edges becomes slightly dull, nicked, or damaged, these cutters tend to tear as much or more than they tend to cut. Consequently, in addition to having difficulty removing a suture, a surgeon employing dull surgical scissors may also damage the tissue surrounding the suture when removing the suture. Specialized procedure cutters, such as the arthroscopic suture cutters noted above, offer little remedy to this problem as the only mode of sharpening these prior art suture cutters is with sophisticated and expensive sharpening tools. Such sharpening tools can cost more than the suture cutter itself.

There is thus a need for an improved suture cutter that is kept sharp simply and inexpensively.

### BRIEF DESCRIPTION OF THE DRAWINGS

The accompanying figures, where like reference numerals refer to identical or functionally similar elements throughout the separate views and which together with the detailed description below are incorporated in and form part of the specification, serve to further illustrate various embodiments and to explain various principles and advantages all in accordance with the present invention.

FIG. 1 illustrates a side, elevation view of a suture cutter in accordance with embodiments of the invention.

FIG. 2 illustrates a top, plan view of a suture cutter in accordance with embodiments of the invention.

FIG. 3 illustrates a side, elevation, cut-away view of a suture cutter in accordance with embodiments of the invention.

FIG. 4 illustrates a top, plan, transparent view of a suture cutter in accordance with embodiments of the invention.

FIG. 5 illustrates a side, elevation view of a suture cutter having its cutting rail in the open position to facilitate a blade change in accordance with embodiments of the invention.

FIG. 6 illustrates a magnified, side, elevation view of a suture hook distally located from a handle in a suture cutter in accordance with embodiments of the invention with a suture disposed therein.

FIG. 7 illustrates a magnified, top, plan view of a suture hook distally located from a handle, in a suture cutter in accordance with embodiments of the invention, upon cutting a suture.

FIG. 8 illustrates a magnified, side, elevation view of a suture hook distally located from a handle of a suture cutter, with the blade in an extended position, in accordance with embodiments of the invention.

FIG. 9 illustrates a method of cutting a suture with an apparatus in accordance with embodiments of the invention.

FIG. 10 illustrates a method of changing a blade in an apparatus in accordance with embodiments of the invention.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present invention.

### DETAILED DESCRIPTION OF THE INVENTION

Embodiments of the invention are now described in detail. Referring to the drawings, like numbers indicate like parts throughout the views. As used in the description herein and throughout the claims, the following terms take the meanings explicitly associated herein, unless the context clearly dictates otherwise: the meaning of “a,” “an,” and “the” includes plural reference, the meaning of “in” includes “in” and “on.” Relational terms such as first and second, top and bottom, and the like may be used solely to distinguish one entity or action from another entity or action without necessarily requiring or implying any actual such relationship or order between such entities or actions. Also, reference designators shown herein in parenthesis indicate components shown in a figure other than the one in discussion. For example, talking about a device (10) while discussing figure A would refer to an element, 10, shown in figure other than figure A.

Embodiments of the present invention provide an apparatus for cutting sutures that has an easily removable and replaceable blade. The blade mounts on an extension rail that is both slidably and rotatably coupled to a handle. When the extension rail is rotated to an open position, the blade can be easily removed. A new blade can then be installed. In one embodiment, a number ten or number fifteen scalpel blade can be used as the cutting edge. Such blades are inexpensive and are readily found in hospitals, ambulatory surgical centers and other operating facilites. The replaceable blade ensures that the cutting apparatus can be maintained in a sharp condition simply and cost effectively.

In one embodiment, the cutting apparatus includes a handle and an extension member. The extension member extends distally from the handle and includes a first extension rail and a second extension rail. In one embodiment, the extension member is sufficiently long as to allow the surgeon to easily reach the sutured region without unnecessarily stretching, contorting, or bending. The first extension rail, in one embodiment, is fixed relative to the handle. The second extension rail is moveable relative to the first extension rail. The second extension rail carries the replaceable blade. The second extension rail is configured to move slidably along the first extension rail by way of a push button. Alternatively, the as noted in the preceding paragraph, the second extension rail can be rotated radially away from the first extension rail to change the detachable blade.

In one embodiment, the extension member includes a suture hook. The suture hook can be used to perform multiple functions. First, in one embodiment, the suture hook can be used to grasp the suture—or thread extending from a suture knot—and slightly pull it away from the tissue in which it resides or the knot from which it extends. Usage of the suture hook in this mode facilitates one-handed use of the cutting apparatus. Whether the suture is being installed or removed, the suture hook allows the surgeon to catch and snip the suture thread with only one hand, thereby freeing up the other.

Second, the suture hook provides a holding apparatus that induces a tension upon the suture. This tension facilitates cutting as the removable blade passes through the tensioned suture. Third, in one embodiment the suture hook defines an aperture into which the detachable blade passes.

A surgeon uses the apparatus in the following fashion: First the surgeon grasps the handle, which in one embodiment resembles a pistol-style grip. If using the apparatus in a one-handed mode, the surgeon then catches the suture or the suture thread with the suture hook such that the suture material across the blade receiving aperture in the suture hook. The surgeon then depresses the push button, thereby causing the blade to pass into the blade receiving aperture, thereby severing the suture material.

Embodiments of the present invention offer many advantages over prior art suture cutters. First and foremost, as noted, the blade can easily be replaced. In one embodiment, the blade is retained by a spring loaded cantilever arm and a corresponding hook. The surgeon, or an assistant can change the blade simply by rotating the extension rail holding the blade open, detaching the old blade and attaching a new blade. As such, the risk of tearing a suture, suture material, or surrounding tissue is greatly mitigated.

Second, since the blade can be changed quickly, a new blade can be used with each patient. Rather than using the same cutting edges found in prior art suture cutters, a clean, sterile, sharp, new blade can be used on each individual, thereby reducing the risk of cross-contamination.

Third, there is no need for sophisticated, expensive sharpening tools. Rather than having to use an expensive, rotary or grinding sharpening tool, one keeps the blade sharp with simple replacement.

Fourth, an apparatus in accordance with the present invention facilitates simple, comfortable one-handed use of the instrument. The pistol-style grip facilitates a confident, full-hand grip while easing fatigue, strain, and other issues.

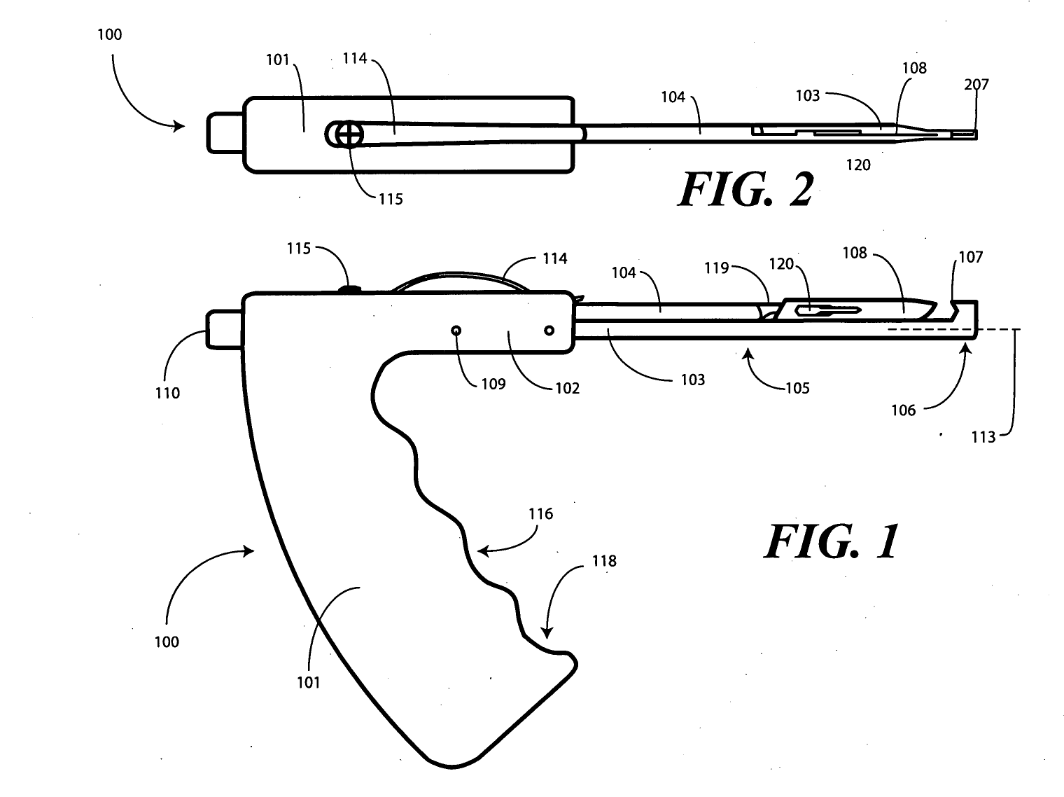

Turning now to FIGS. 1 and 2, illustrates therein is one embodiment of an apparatus for cutting sutures in accordance with embodiments of the invention. The apparatus 100 includes a handle 101 having a support member 102 extending therefrom. The support member 102 holds a first extension rail 103 and a second extension rail 104. The two extension rails 103,104 collectively form an extension member 105 that is coupled to the handle 101, such as by way of the support member 102. In one embodiment, the extension member is between five and ten inches in length. Experimental testing has shown that an extension member length of approximately eight inches provides a nice balance of reach—for cutting sutures, regardless of location on the patient, or for cutting excess suture material while suturing—and ease of handling.

The extension member 105 extends distally from the handle 101 and terminates in a suture hook 106. The suture hook 106 includes a hooked member 107 that can be used to catch a suture or excess suture material when the apparatus 100 is used in a one-handed operating procedure. In one embodiment, the suture hook 106 also defines a blade receiving aperture 207 into which the blade 108 passes when cutting the suture. In the embodiment of FIGS. 1 and 2, the blade receiving aperture 207 passes through the hooked member 107 such that the blade receiving aperture 207 is visible from the top (FIG. 2) of the apparatus 100. This configuration is optional, however, as the hooked member 107 can be configured to pass over the top of the blade 108 such that the blade receiving aperture 207 is non-visible from the top of the apparatus 100.

The first extension rail 103, in one embodiment, is coupled fixedly with the handle 101. It is coupled fixedly in that it does not move vertically or horizontally relative to the handle 101 when cutting sutures. However, the first extension rail 103 can be configured to be detachable from the handle 101. Where a detachable handle is used, the extension member 105 can be switched from handle to handle such that smaller handles may be affixed to the extension member 105 for surgeons with smaller hands. Alternatively, larger handles may be attached to the extension member 105 for surgeons with larger hands. Further, shorter or longer extension rails can be coupled to the handle 101 for different applications. In such a configuration, the extension member 105 may be detachable, yet fixedly coupled to the handle 101 with a fastener, such as with the number three cap screws 109 shown in FIG. 1.

The suture hook 106 and hooked member 107 are coupled to the first extension rail 103. In one embodiment, the first extension rail 103 and suture hook 106 are formed as a unitary member, such as by milling metal, forging, or injection molding. Where sutures of vastly different sizes are to be cut, the suture hook 106 can be detachable from the first extension rail 103. In such an embodiment, the suture hook 106 may be fixedly coupled to the first extension rail 103 by a fastener, such as a screw or bolt. Alternatively, the suture hook 106 may be threaded, and may affix to the first extension rail 103 accordingly. To facilitate ease of catching sutures, the suture hook 106 may be thinner in diameter than, for instance the first extension rail 103. For example, the suture hook 106 may only be 0.075 inches across, while the first extension rail 103 may be three or more times that width.

The second extension rail 104 is configured to be moveable relative to the first extension rail 103. When cutting sutures or suture material, the second extension rail 104 moves laterally parallel to a lengthwise axis 113 of the first extension rail 103. In the illustrative embodiment of FIGS. 1 and 2, a push button 110 is coupled to the second extension rail 104 to facilitate simple, ergonomic actuation of the second extension rail 104 along the first extension rail 103. As the action of the push button 110 is described as one illustrative embodiment, it will be clear to those of ordinary skill in the art having the benefit of this disclosure that the invention is not so limited. Other devices can be substituted for the push button 110, including a slider coupled to the side of the handle 101 or a trigger extending from the handle 101. The slider and trigger are shown as dashed lines as the illustrative embodiment of FIGS. 1 and 2 employs the push button 110.

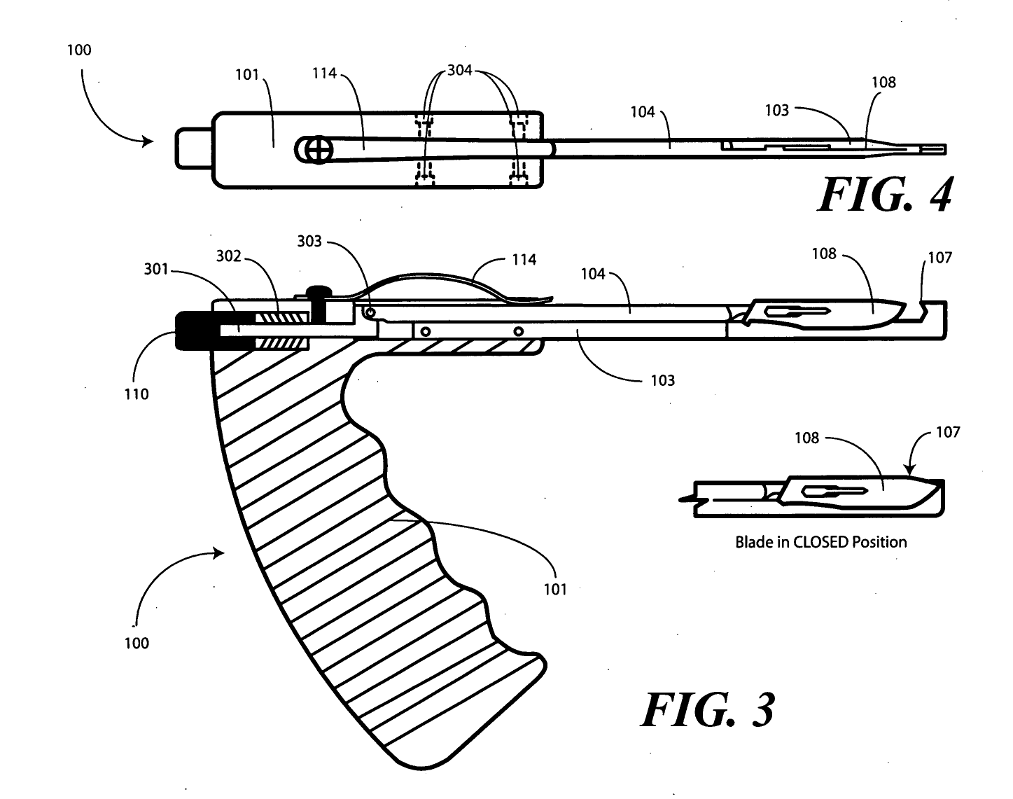

In one embodiment, the push button 110 is coupled with the second extension rail 104 within the support member 102 (this will be shown in more detail in the discussion of FIGS. 3 and 4) and extends outwardly from the back of the handle 101. The push button 110 is located such that it may be conveniently actuated by a user's thumb when the user grasps the handle.

The push button 110 is configured such that, when actuated by a user, it causes the second extension rail 104 to move laterally relative to the first extension rail 103 away from the handle 101. When the push button 110 is fully depressed, the second extension rail 104 moves sufficiently far that the blade 108 passes into the blade receiving aperture 207. Where a suture is within the hooked member 107, the suture is thereby cut when the blade 108 passes into the blade receiving aperture 207.

As noted above, one advantage of the apparatus 100 of FIGS. 1 and 2 is that the blade 108 is detachable. To facilitate simple blade replacement, in one embodiment the second extension rail 104 is not only configured to be slideable along the first extension rail 103, but is configured to be rotatable away from the first extension rail 103 as well. When blade replacement is not required, a compliance member 114 is used to bias the second extension rail 104 against the first extension rail 103.

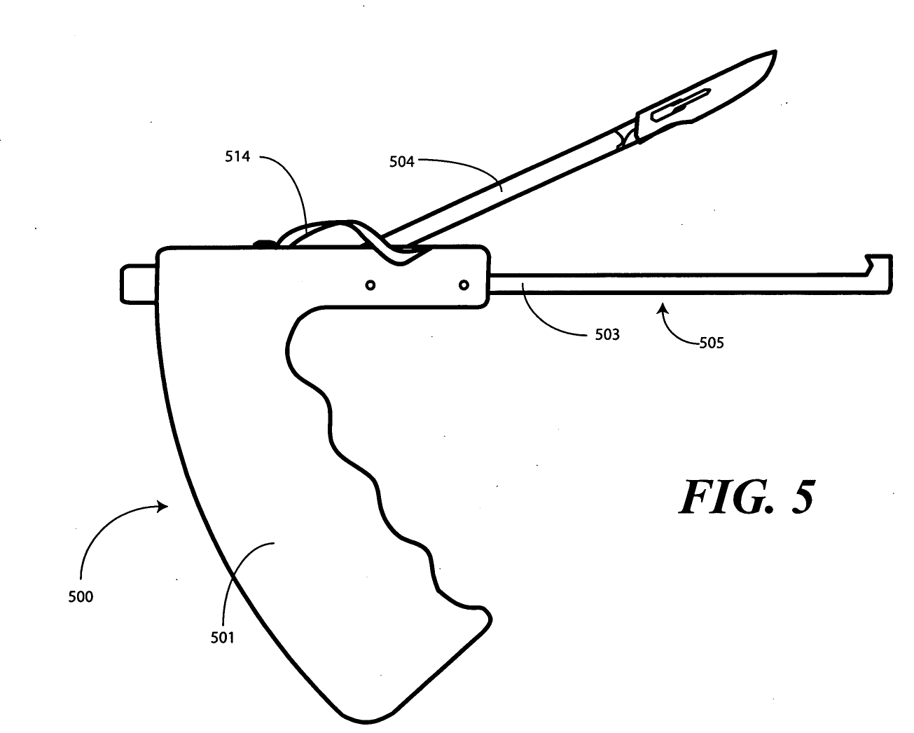

In the illustrative embodiment of FIGS. 1 and 2, the compliance member 114 is configured as an arched leaf spring coupled to the handle by a fastener 115, shown in FIGS. 1 and 2 as a screw. As will be shown in more detail in FIG. 5, the compliance member 114 is moveable from a first compliance member position (shown in FIGS. 1 and 2) to a second compliance member position (shown in FIG. 5). When in the first compliance member position, the compliance member 114 applies a biasing force against the second extension rail 104 so as to retain the second extension rail 104 in a substantially parallel configuration with the first extension rail 103. The term “substantially” is used because the two extension rails 103,104 need not be perfectly parallel for proper operation. Slight angles due to manufacturing tolerances or compliance member age will not affect the operation. When in the second compliance member position (shown in FIG. 5) the biasing force against the second extension rail 104 is removed so that the second extension rail 104 can be rotated radially away from the first extension rail 103 to facilitate blade replacement.

In one embodiment, the handle 101 is configured as a pistol-style grip. Experimental testing has shown that such a grip facilitates comfortable and convenient apparatus operation. By way of example, the illustrative handle 101 of FIGS. 1 and 2 is configured as a pistol grip with a finger receiving surface 116 along a first edge of the pistol grip. The finger receiving surface 116 includes a plurality of finger recesses 118 into which the user's fingers can be placed for consistent hand-apparatus alignment. A palm receiving surface 117 is disposed along a second edge—disposed opposite the first edge—of the pistol grip to further facilitate this consistent, comfortable alignment. In the illustrative embodiment of FIGS. 1 and 2, the palm receiving surface 117 comprises a convex contour. It will be clear to those of ordinary skill in the art having the benefit of this disclosure that the invention is not so limited, however, as a pistol grip is but one style of grip that can be used with embodiments of the invention.

In the illustrative embodiment of FIGS. 1 and 2, the extension member extends from the pistol grip at an angle of between 30 and 150 degrees. In particular, angles of between 45 and 90 degrees have been shown to be effective in use. This is because the apparatus 100 may often be used in a generally vertical orientation, such as when installing or removing sutures. As such, an angle of about 60-65 degrees, as shown in FIG. 1, facilitates a comfortable grip and easy thumb-push button access when the apparatus 100 is in the vertical orientation.

The detachable blade 108, in one embodiment is coupled to the second extension member 104 by a spring clip 120 and hook 119. To change the blade 108, a user simply slides the blade 108 away from the handle 108 such that the hook 119 releases the blade 108 and the aperture in the blade 108 clears the spring clip 120. Attaching the blade 108 is the process in reverse. In one embodiment, the blade 108 is configured to be either a number ten or number fifteen scalpel blade, as such blades are readily available in surgical centers. Further, these blades are relatively inexpensive. While a spring clip 120 is one way to attach the blade 108, it will be clear to those of ordinary skill in the art having the benefit of this disclosure that the invention is not so limited. Other blade attaching means, including notch and hook couplings, screws, and the like may also be used.

Turning now to FIGS. 3 and 4, illustrated therein are cutaway and transparent views of a cutting apparatus 100 in accordance with embodiments of the invention. The views of FIGS. 3 and 4 allow the internal components of one illustrative embodiment of the apparatus 100 to be seen.

First, the coupling of the push button 110 and the second extension rail 104 can be seen. In FIG. 3, the push button 110 is coupled to the second extension rail 104 by a linkage component 301. A compliance member 302, which is disposed between the handle 101 and the push button 110 then biases the push button 110 into an extended position from the handle 101. From another perspective, the compliance member 302 also biases the detachable blade 108 towards the handle 101. In the illustrative embodiment of FIG. 3, the compliance member 302 is a coil spring, such as a compression spring measuring 0.35×0.360×0.750 inches.

Next, the “rotatable” capability of the second extension member 104 can be seen in FIG. 3. In this illustrative embodiment, the second extension rail 104 is coupled to the push button 110—via the linkage component 301—by way of a pin 303. The second extension rail 104 is configured to rotate about the pin 303 when the compliance member 114 is pivoted away from the second extension member 104. When the second extension rail 104 moves about the pin 303, the detachable blade 108 moves radially away from, or towards, the hooked member 107. By moving the second extension rail 104 radially away from the hooked member 107, a user may replace the blade 108. By moving the second extension rail towards the hooked member 107, and latching it in place with the compliance member 114, the user makes the apparatus 100 ready for cutting. Note that in this illustrative embodiment, the compliance member 114 is held to the handle 101 by a number 6−32×0.250 cap screw.

In FIG. 4, the coupling mechanism of the first extension rail 103 to the handle 101 can be seen. In this illustrative embodiment, screws 304 are used. In particular, number 3−48×0.250 cap screws are used to affix the first extension rail 103 to the handle 101.

Assembly in manufacture, as well as field stripping, of the apparatus 100 is relatively simple. One begins assembling the apparatus by first sliding the second extension rail 104 through the handle 101. The compliance member 302 is then passed into the handle 101 about the linkage component 301, where one is used. The push button 110 is then adhesively or otherwise affixed to the linkage component 301. In one embodiment, both the linkage component 301 and the push button 110 are threaded to assist in retention.

From this point, the first extension rail 103 can be affixed to the handle 101 using cap screws or other suitable means. Adhesive can be used to retain the cap screws within the handle 101. Compliance member 114 can be installed by way of a screw or other means. Disassembly is this process in reverse.

Turning now to FIG. 5, illustrated therein is a suture cutter 500 in accordance with embodiments of the present invention having a moveable, second extension rail 504 rotated to an open position to facilitate blade replacement. The compliance member 514 has been rotated to the second compliance member position such that the second extension rail 504 can be selectively rotated away from the first extension rail 503, which represents an elongated member 505 extending distally from the handle 501. In this configuration, the blade 508 can easily be replaced.

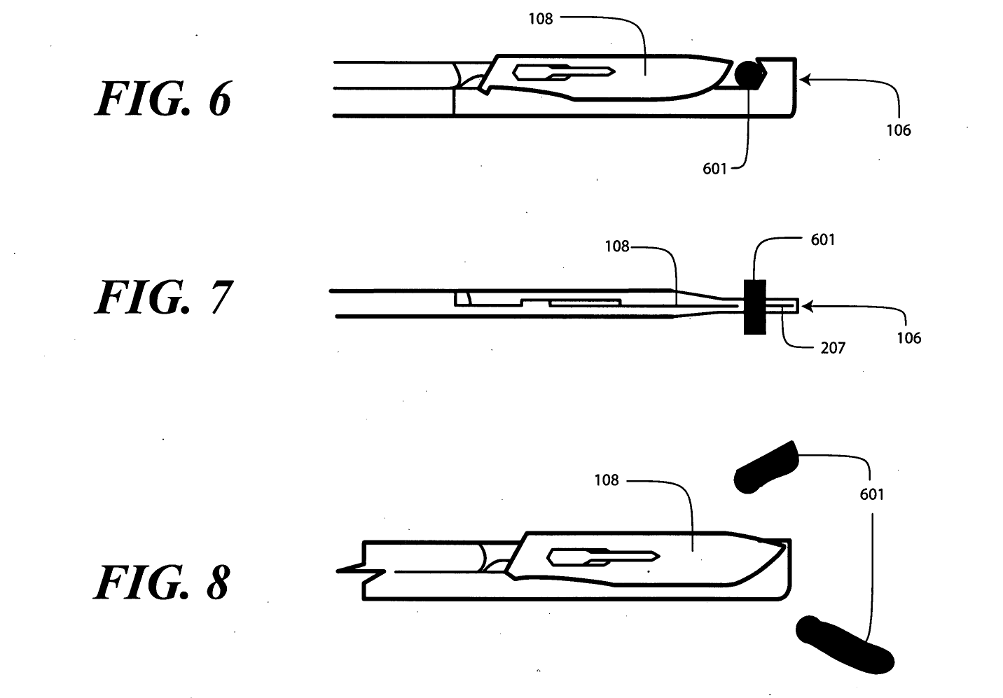

Turning now to FIGS. 6, 7, and 8 illustrated therein are magnified elevation and plan views of the suture hook 106. FIG. 6 illustrates a side, elevation view before the blade 108 is actuated. FIG. 7 illustrates a top, plan view. FIG. 8 illustrates a side, elevation view after the blade 108 has been actuated. In FIGS. 6 and 7, unsevered suture material 601 rests within the suture hook 106. In FIG. 8, the suture material 601 has been severed due to actuation of the blade 108. When the blade 108 is actuated, the movable, second extension rail (104) moves along a longitudinal axis (113) of the first extension rail (103) such that the blade 108 enters the slotted, blade receiving aperture, thereby cutting the suture material 601.

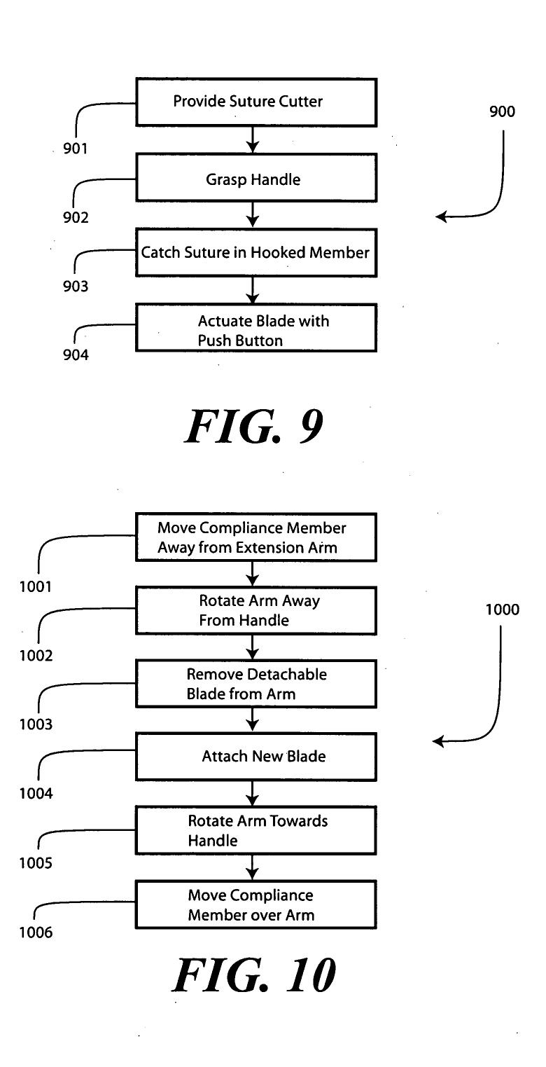

Turning now to FIG. 9, illustrated therein is one method 900 of cutting a suture (601) in accordance with embodiments of the invention. At step 901, an apparatus (100) for cutting suture material is provided. The suture cutter includes a handle (101) and an extension member (e.g. extension member 105) extending distally therefrom. The extension member includes a hooked member (107) disposed distally from the handle (101). The hooked member (107) defines a blade receiving aperture (207) therein. The extension member also includes a moveable extension rail (104) having a blade (108) detachably coupled thereto. In one embodiment, the moveable extension rail (104) is configured as to be moveable via the actuation of a push button (110) that is coupled to the moveable extension rail (104).

At step 902, the user grasps the handle (101) of the apparatus (100). Where the handle (101) is configured as a pistol grip, the user wraps their fingers about the pistol grip such that the hand rests comfortably on the pistol grip and the thumb has easy access to the push button (110). At step 903, the user catches suture material (601) with the hooked member (107). Where necessary upon catching the suture material (601), the user may then move or rotate the apparatus (100) slightly upon catching the suture material (601) so as to cause the suture material (601) to pass across the blade receiving aperture (207). At step 903, the user actuates the push button (110), thereby causing the blade (108) to pass into the blade receiving aperture (207). When the blade (108) passes into the blade receiving aperture (207), the suture material (601) is cut or severed.

In one embodiment, the moveable extension rail (104) is pivotally coupled to the suture cutter to facilitate simple blade replacement. In such an embodiment, and the suture cutter may further have a compliance member (114) configured to retain the moveable extension rail (104) in a first, closed, radial orientation. Turning now to FIG. 10, illustrated therein is one method of changing a blade with such a suture cutter.

At step 1001, the user moves the compliance member (114) from a first compliance member position, i.e., with the compliance member applying a force to the moveable extension member (104), to a second compliance member position. The second compliance member position is such that the compliance member (114) has been adjusted such that the moveable extension rail (104) can pivot freely away from the extension member.

With the compliance member (114) moved out of the way, the user may then rotate the moveable extension rail (104) from a first radial extension rail position to a second radial extension rail position at step 1002. The first radial extension rail position occurs where the moveable extension rail (104) is substantially parallel with the extension member. The second radial extension rail position is where the moveable extension rail (104) has been rotated such that an angle of about 15 degrees or more is defined by the moveable extension rail (104) and the extension member. This movement causes the blade (108) to move radially away from the hooked member (107). At step 1003, the user detaches the blade (108) from the moveable extension rail (104).

The user may attach a new, sharp blade just as simply. At step 1004, the user couples a second, new, sharp blade to the moveable extension rail (104). The user then rotates the moveable extension rail (104) from the second radial extension rail position to the first radial extension rail position at step 1005. At step 1006, the user moves the compliance member from the second compliance member position to the first compliance member position. The suture cutter is now ready to use as shown in FIG. 9.

In the foregoing specification, specific embodiments of the present invention have been described. However, one of ordinary skill in the art appreciates that various modifications and changes can be made without departing from the scope of the present invention as set forth in the claims below. Thus, while preferred embodiments of the invention have been illustrated and described, it is clear that the invention is not so limited. Numerous modifications, changes, variations, substitutions, and equivalents will occur to those skilled in the art without departing from the spirit and scope of the present invention as defined by the following claims. Accordingly, the specification and figures are to be regarded in an illustrative rather than a restrictive sense, and all such modifications are intended to be included within the scope of present invention. The benefits, advantages, solutions to problems, and any element(s) that may cause any benefit, advantage, or solution to occur or become more pronounced are not to be construed as a critical, required, or essential features or elements of any or all the claims.

What is claimed is:

1. An apparatus for cutting sutures, comprising:
a handle;
an extension member coupled to the handle and extending distally from the handle, the extension member comprising:
a first extension rail coupled fixedly with the handle, wherein the first extension rail comprises a hooked member defining a blade receiving aperture therein and coupled to the first extension rail distally from the handle; and
a second extension rail, configured to be moveable relative to the first extension rail; and
a detachable blade coupled to the second extension rail distally from the handle; and
a push button coupled with the second extension rail and extending from the handle, wherein the push button is configured to cause the second extension rail to move laterally relative to the first extension rail, away from the handle, upon actuation of the push button, such that the detachable blade passes into the blade receiving aperture.

2. The apparatus of claim 1, further comprising a compliance member disposed between the handle and the push button so as to bias the detachable blade towards the handle.

3. The apparatus of claim 2, wherein the compliance member comprises a coil spring.

4. The apparatus of claim 1, wherein the handle comprises a pistol grip.

5. The apparatus of claim 4, wherein the pistol grip comprises a finger-receiving surface along a first edge of the pistol grip, the finger-receiving surface defining a plurality of finger recesses.

6. The apparatus of claim 5, wherein the pistol grip comprises a palm-receiving surface along a second edge of the pistol grip, the second edge disposed opposite the first edge, the palm-receiving surface defining a convex contour along the second edge.

7. The apparatus of claim 4, wherein the extension member extends from the pistol grip at an angle of between 30 and 150 degrees.

8. The apparatus of claim 7, wherein the extension member extends from the pistol grip at an angle of between 45 and 90 degrees.

9. The apparatus of claim 1, wherein the second extension rail is coupled to the push button by a pin such that the second extension rail is configured to rotate about the pin, whereby upon rotation of the second extension rail about the pin, the detachable blade moves radially relative to the hooked member.

10. The apparatus of claim 9, further comprising a compliance member, configured to be moveable from at least a first compliance member position to at least a second compliance member position, wherein the compliance member is configured such that when in the first compliance member position the compliance member applies a biasing force against the second extension rail so as to retain the second extension rail in a substantially parallel configuration with the first extension rail.

11. The apparatus of claim 10, wherein when the compliance member is configured such that when in the second compliance member position, the biasing force against the second extension rail is removed.

12. The apparatus of claim 10, wherein the compliance member comprises a leaf spring.

13. The apparatus of claim 1, wherein the detachable blade is coupled to the second extension rail by one of notch and hook coupling, a spring clip, or one or more screws.

14. The apparatus of claim 1, wherein the detachable blade comprises one of a number ten scalpel blade or a number fifteen scalpel blade.

15. A method of cutting suture material, the method comprising the steps of:
providing a suture cutter comprising a handle and an extension member extending distally therefrom, the extension member comprising a hooked member disposed distally from the handle and defining a blade receiving aperture therein and a moveable extension rail having a blade detachably coupled thereto, the moveable extension rail configured as to be moveable by actuation of a push button coupled thereto;
grasping the handle;
catching the suture material with the hooked member such that the suture material passes across the blade receiving aperture; and
actuating the push button, thereby causing the blade to pass into the blade receiving aperture, thereby severing the suture material.

16. The method of claim 15, wherein the moveable extension rail is pivotally coupled to the suture cutter and the suture cutter further comprises a compliance member configured to retain the moveable extension rail in a first radial orientation, further comprising the steps of:
moving the compliance member from a first compliance member position to a second compliance member position;
rotating the moveable extension rail from a first radial extension rail position to a second radial extension rail position, thereby moving the blade radially away from the hooked member; and
detaching the blade from the moveable extension rail.

17. The method of claim 16 further comprising the steps of:
coupling a second blade to the moveable extension rail;
rotating the moveable extension rail from the second radial extension rail position to the first radial extension rail position; and
moving the compliance member from the second compliance member position to the first compliance member position.

18. A suture cutter configured for one handed operation, the suture cutter comprising:
a handle having an elongated member extending distally therefrom, wherein the elongated member comprises:
a suture hook defining a slotted aperture therein, the suture hook being disposed distally from the handle;
a movable rail having a blade detachably coupled thereto distally from the handle, wherein the movable rail is pivotally coupled to the suture cutter so as to be selectively rotatable away from the elongated member; and
a thumb actuation switch coupled to the movable rail and configured to cause the movable rail to move along a longitudinal axis of the elongated member upon actuation such that the blade enters the slotted aperture.

19. The suture cutter of claim 18, further comprising a retention device configured to retain the movable rail along the elongated member when in a retention position.

20. The suture cutter of claim 18, wherein the handle comprises a pistol grip configured for engagement with a user's hand, the pistol grip defining a plurality of finger recesses thereon and extending along a curvature from the elongated member.
